(19) United States
(12) Patent Application Publication
(10) Pub. No.: US 2024/0091310 A1
(43) Pub. Date: Mar. 21, 2024

(54) COMPOSITIONS AND METHODS USEFUL FOR THE PREVENTION AND/OR TREATMENT OF DISEASE IN MAMMALS

(71) Applicant: VIRO-GEN (PTY) LTD, Pretoria, Gauteng (ZA)

(72) Inventor: Michelle Olga Patricia Giesteira Da Silva SMIT, Pretoria (ZA)

(21) Appl. No.: 18/004,631

(22) PCT Filed: Jul. 9, 2021
(86) PCT No.: PCT/IB2021/056179

§ 371 Date: Jan. 6, 2023

(30) Foreign Application Priority Data
Jul. 9, 2020 (ZA) 2020/03850
Jul. 9, 2020 (ZA) 2020/04008

(51) Int. Cl. A61K 38/18; A61K 31/16

(52) U.S. Cl. CPC A61K 38/1825; A61K 31/16; A61K 48/00

(57) ABSTRACT

Novel methods and pharmaceutical composition or medicaments are described for protecting a subject against, or treating a subject suffering from, disease characterised by viral infection and/or diseases associated with immune system disorder and/or viral cancers, by raising the levels of Ksp37 in the blood plasma of the subject to a therapeutically effective concentration level. According to the invention, a therapeutically effective amount of one or more of; a clinically modified or genetically engineered Ksp37 protein and/or proteins having a molecular weight in the range ranging from 24 kDa to 45 kDa, and/or a vector encoded with a KSP37 gene which will translate to a Ksp37 protein and/or proteins having a molecular weight in the range ranging from 24 kDa to 45 kDa, and/or a polar compound are administered to a subject.

### CROSS REFERENCE TO RELATED APPLICATIONS

This application is a U.S. National Stage Application under 35 U.S.C. 371 of International Application No. PCT/IB2021/056179, filed Jul. 9, 2021, which claims priority to South African Application Nos. ZA202003850 and ZA202004008, filed Jul. 9, 2020, the content of each of which is incorporated by reference in its entirety into the present disclosure.

### FIELD OF THE INVENTION

This invention relates to compositions and methods useful for the treatment of disease in mammals, characterised by viral infection and/or diseases associated with immune system disorders and/or viral cancers.

### BACKGROUND TO THE INVENTION

The virus known as Human Immunodeficiency Virus (“HIV”), classified as a retrovirus, has affected the lives of millions of people across the world. The virus infects healthy individuals who progress to developing Acquired Immunodeficiency Syndrome (“AIDS”) within a few years if no treatment is administered (Mann, J. et al. (2016) ‘The latest science from the IAS Towards an HIV Cure Symposium 16-17 Jul. 2016, Durban, South Africa’, (July), pp. 235-241.).

HIV is a member of the lentivirus family of retroviruses (Teich et al., 1984, RNA Tumor Viruses, Weiss et al., eds., CSH-press, pp. 949-956). Retroviruses are small enveloped viruses that contain a single-stranded RNA genome, and replicate via a DNA intermediate produced by a virally-encoded reverse transcriptase, an RNA-dependent DNA polymerase (Varmus, H., 1988, Science 240:1427-1439).

Other retroviruses include, for example, oncogenic viruses such as human T-cell leukemia viruses (HTLV-1, -II, -III), and feline leukemia virus.

HIV Structure and Genome Organisation

Mature HIV virions are 100 nm-120 nm in diameter spherical structures consisting of a lipid bilayer membrane which encloses a dense truncated cone-shaped nucleocapsid (“core”). The core contains two 9.8 kb long positive sense, single stranded, linear RNA molecules, molecules to initiate cDNA synthesis, cellular tRNA, Gag polyprotein, viral envelope (Env) protein and three enzymes: reverse transcriptase (RT), viral protease (PR), integrase (IN), and some other cellular factors (S Sierra, et al., 2005).

The HIV genome contains accessory and regulatory genes flanked by long terminal repeats (“LTR”). The viral genome has a total of nine genes which can be divided into three functional groups:
structural genes, Gag, Pol, and Env;
regulatory genes, Tat and Rev; and
accessory genes, Vpu, Vpr, Vif, and Nef (J M Costin, 2007).

The Gag gene codes for the core protein, Pol gene codes for RT, protease, integrase, and Env gene codes for the Envelope protein (gp160). The Tat gene codes for the Tat protein and the Rev gene codes for the Rev protein. The Tat and Rev regulatory proteins function as RNA-binding proteins. In addition to RNA binding, Tat proteins also act as activators of transcription ensuring that full length genomes of HIV are formed. Rev protein also helps in shift of gene expression of HIV from early to late phase. On the other hand, the accessory proteins coded for by the accessory genes, are multifunctional. Nef or negative factor is involved in T-cell activation, down-regulation of existing major histocompatibility complex (MHC) I, and CD4 on the cell surface by degranulation in lysosomes and also stimulate virion infectivity. Vpr acts as a nucleo-cytoplasmic transport factor which permits HIV to infect non-dividing cells. Vpu enhances release of virion through the development of an ion channel and also down-modulates expression of CD4 through ubiquitin-mediated degradation. Replication of HIV in lymphocytes, monocytes, and macrophages is regulated by Vif.

The envelope of the virion contains the transmembrane proteins, gp120 and gp41, which project outwards from the virion in the form of spikes (up to 72 in number). Being a highly immunogenic protein, gp120, which binds to the CD4 receptor, is a suitable target for majority of host antibodies.

Most of these strain-specific antibodies block the interaction of CD4 receptors with gp120 protein by binding to these receptors. The matrix lying underneath the lipid bilayer consists of Gag protein 17 (viral gag protein cleavage product). The core or capsid contains a covering of p24 protein (product of Gag gene), and a third Gag protein p7 (Lampejo T et al., 2013).

HIV Life Cycle

Human immunodeficiency virus viral entry is divided into basically three steps:
(1) binding;
(2) activation; and
(3) fusion.

Major HIV-1 and HIV-2 receptors and co-receptors are CD4 and CCR5, CXCR4, respectively. The cycle starts with the recognition of HIV-enveloped trimeric complex, gp120 and gp41 with CD4 receptor (58 kDa monomeric glycoprotein) major co-receptor of MHC class II molecule, on cell surface. Upon binding of CD4 with gp120, a conformational change occurs resulting in exposure of gp120 domain where CCR5 chemokine co-receptors bind. So far, 17 chemokine receptor ligands are identified in this process (Fanales-Belasio et al., 2010).

Following double binding of gp120, a stable attachment complex formed which allows the N-terminal side of gp120 peptide penetration in plasma membrane. In gp41 protein, HR1 and HR2 sequences act together and form a hairpin structure of gp41, which causes fusion of viral and cellular membranes (S Sierra et al., 2005).

After fusion viral core is released in cytoplasm, uncoating of viral capsid occurs mediated by MA, Nef, and Vif protein factors of virus (Lampejo T et al., 2013).

By viral RT ribonuclease H site viral RNA is transcribed into DNA starting from primer binding site. After completion of transcription, ribonuclease H breaks the dsRN/DNA hybrid and by RT polymerization active site converted into dsDNA (Fanales-Belasio et al., 2010). Proviral status is obtained by integration of this dsDNA into host cell genome by integrase enzyme. The integrase protein produces sticky ends at 3′ end of each DNA strand. Now modified viral DNA is exported to nucleus through nuclear pore, directed by viral Vpr, and integration function is accomplished by this integrase (Sierra S).

For the viral genome to be expressed, the host genome integration site should be in active state (Fanales-Belasio).

In the provirus state, the viral DNA may remain for several years in host genome and upon receiving activation signal express mRNA using host polymerase enzyme (Yousaf M Z et al., 2011).

Latently infected T cells, macrophages, monocytes, and microglial cells are major reservoirs of HIV genome. In active cell state, transcription of HIV genome starts due to host RNA polymerase II and other transcription factors by binding with viral LTRs. Following transcription, translation results in basal amount of proteins (Tat, Rev and Nef). On adequate production of Tat, further transcription is controlled by binding of Tat with TAR elements on LTRs and other transcriptional cellular activators (Sierra S).

In early stages, multiply spliced mRNA produces Rev, Tat, and Nef. On achieving adequate amounts of Rev, non-spliced and longer mRNAs are produced, referred to polysome, resulting in the production of other viral proteins and genomic RNA. On the un-spliced RNA RRE, Rev response elements are present where Rev binds and causes the safe transportation, without splicing, to cell cytoplasm for translation (Lampejo T).

REV also causes expression of enzymatic and structural proteins and regulatory proteins inhibition so plays a role in producing mature virion. In cytoplasm, ENV gene is translated into gp160 glycosylated in ER resulted into mature gp120 and gp140 by HIV-1 protease (Fanales-Belasio).

During translation, ribosome-1 frame shift resulting in Gag pol proteins includes PR, RT, and IN. Nucleus of mature virions are formed by Gag and Pol gene proteins. From large 160 kDa precursor Gag and Pol proteins are formed cleaved by viral proteases into p24, p9, p7, p17 Gag final products and Pol products. This cleavage is necessary for infectious viral particles ENV proteins, which after translation move toward membrane and is inserted into it. Gag and Gag-Pol polyprotein also move toward cell membrane and started to assemble mediated by Gag polyprotein. Full size genomic RNA, cellular tRNAlys-3-primer, enzymes, and all cellular compounds become linked with immature viral core (Sierra S).

Budding of immature virus takes place through plasma membrane. It is necessary to have a reduced number of CD4 molecules on the cell surface when virus assembly and budding occurs. Nef, ENV, and Vpu are involved in this process. Nef in early stages mediate the endocytosis and mortification of MHC class I and II molecules. In later stages, Npu induces the degradation of CD4 molecules. During budding, activation of protein protease takes place which auto-catalytically cleaves Gag and Gag-Pol polyprotein resulted in structural proteins and viral enzymes. Further interactions of individual proteins with capsid, nucleocapsid protein resulted in conic nucleocapsid, and MA remain associated to viral envelop (Sierra S).

Current Treatment for HIV Infection

The current treatment for HIV infection involves a combination of:
Non-nucleoside reverse transcriptase inhibitors;
Nucleoside reverse transcriptase inhibitors; and
Protease inhibitors.

This combination of drugs is commonly referred to as Highly Active Anti-Retroviral Treatment (“HAART”). HAART is provided to patients with the goal of slowing down the AIDS development, by inhibiting the viral DNA from being incorporated into the host DNA, and also by inhibiting the formation of viral DNA from viral RNA (Chupradit K et al., 2017 ‘Current peptide and protein candidates challenging HIV therapy beyond the vaccine Era’, Viruses, 9(10), pp. 1-14. doi: 10.3390/v9100281).

These are just a few of the drugs and their mechanisms of action that are given to patients that have tested positive for HIV.

To date, attempts at developing a vaccine against, or cure for, HIV infection have not proven successful.

The typical approach to vaccine development is to infect the body with parts of a virus and elicit an anti-body response. When the vaccinated host becomes infected with the real virus in future, the immune system will recognize the antigen of the virus which was contained in the vaccine, and an immediate overwhelming immune response will be triggered. While this vaccine development strategy works for some viruses, it does not work for the HI-Virus responsible for HIV infection and subsequent AIDS, because of the ability of the HI-Virus to mutate. As a result of the mutation of the HI-Virus, detection of viral antigens is delayed, allowing proliferation of virus-infected cells.

In humans, HIV replication occurs prominently in CD4+T lymphocyte populations, and HIV infection leads to a depletion of this cell type and eventually to immune incompetence, opportunistic infections, neurological dysfunctions, neoplastic growth, and ultimately death.

South African Patent 1998/04649 entitled “Drug Delivery Devices and Methods for Treatment of Viral and Microbial Infections and Wasting Syndromes”, teaches a drug delivery device for transdermal administration of a therapeutic agent, comprising a reservoir containing or having absorbed thereon a therapeutic composition comprising the polar compound N,N-dimethylformamide (“DMF”).

Individuals, the study of whom generated the teachings of ZA 1998/064649, despite being infected with HIV have maintained a CD4⁺ T-cell count above 350 for more than 20 years without being on antiretroviral treatment. These individuals are referred to herein as Long-term Non-Progressors (“LTNP”).

One such individual, had initially presented with HIV PCR detecting 63.298 HIV-1 RNA copies/ml of plasma in December 1996. A number of years after participation in the transdermal DMF study, a follow-up HIV PCR conducted in March 2006, detected <40 HIV-1 RNA copies/ml of plasma; and even more encouraging, in 2011 a further follow-up HIV PCR detected No HIV-1 RNA copies/ml of plasma and a CD4 Count of 920/μL.

The mode of action of the DMF was not fully understood, but further studies on this group of LTNP individuals revealed that their CD8⁺ cells produce elevated levels of a protein known as Ksp37.

The characteristics of the CD8⁺ cells that produce Ksp37 include:
Phenotype type markers for these cells are: CD27, CD45RO, and CD57; and
The signal molecule for these cells has been confirmed to be MIP-1s (Bennett, Salter and Smith, 2018 ‘A New Class of Antiretroviral Enabling Innate Immunity by Protecting APOBEC3 from HIV Vif-Dependent Degradation’, Trends in Molecular Medicine. Elsevier Ltd, 24(5), pp. 507-520. doi: 10.1016fj.molmed.2018.03.004.).

While Ksp37 is present in all humans and many other animals, it is usually present in quantities less than 400 ng/mL. It is hypothesised that the levels of Ksp37 in the LTNP individuals may be a key reason why HIV is unable to progress in these individuals.

Characteristics of Protein Ksp37

The KSP37 gene (also known as FGBP2) is commonly expressed by NK, CD8⁺ T, cd T and CD4⁺ T cells and is composed of 223 amino acids (Ogawa et al., 2001 ‘A Novel Serum Protein That Is Selectively Produced by Cytotoxic Lymphocytes’, The Journal of Immunology, 166(10), pp. 6404-6412. doi: 10.4049fjimmunol.166.10.6404).

The protein known as Ksp37 or Killer-specific secretory protein of 37 kDa, or Fibroblast Growth Factor-Binding Protein 2 (FGF-BP2) has been isolated, and sequenced as follows:

```
ORIGIN
      1 ccctttaaag ggtgactcgt cccacttgtg
          ttctctctcc tggtgcagag ttgcaagcaa
    61 gtttatcaga gtatcgccat gaagttcgtc
          ccctgcctcc tgctggtgac cttgtcctgc
  121 ctggggactt tgggtcaggc cccgaggcaa
          aagcaaggaa gcactgggga ggaattccat
  181 ttccagactg gagggagaga ttcctgcact
          atgcgtccca gcagcttggg gcaaggtgct
  241 ggagaagtct ggcttcgcgt cgactgccgc
          aacacagacc agacctactg gtgtgagtac
  301 agggggcagc ccagcatgtg ccaggctttt
          gctgctgacc ccaaacctta ctggaatcaa
  361 gccctgcagg agctgaggcg ccttcaccat
          gcgtgccagg gggccccggt gcttaggcca
  421 tccgtgtgca gggaggctgg accccaggcc
          catatgcagc aggtgacttc cagcctcaag
  481 ggcagcccag agcccaacca gcagcctgag
          gctgggacgc catctctgag gcccaaggcc
  541 acagtgaaac tcacagaagc aacacagctg
          ggaaaggact cgatggaaga gctgggaaaa
  601 gccaaaccca ccacccgacc cacagccaaa
          cctacccagc ctggacccag gcccggaggg
  661 aatgaggaag caaagaagaa ggcctgggaa
          cattgttgga aacccttcca ggccctgtgc
  721 gcctttctca tcagcttctt ccgagggtga
          caggtgaaag acccctacag atctgacctc
  781 tccctgacag acaaccatct ctttttatat
          tatgccgctt tcaatccaac gttctcacac
  841 tggaagaaga gagtttctaa tcagatgcaa
          cggcccaaat tcttgatctg cagcttctct
  901 gaagtttgga aaagaaacct tcctttctgg
          agtttgcaga gttcagcaat atgataggga
  961 acaggtgctg atgggcccaa gagtgacaag
          catacacaac tacttattat ctgtagaagt
1021 tttgctttgt tgatctgagc cttctatgaa
          agtttaaata tgtaacgcat tcatgaattt
1081 ccagtgttca gtaaatagca gctatgtgtg
          tgcaaaataa aagaatgatt tcagaaat
```

The sequence is saved under the accession number AB021123, for BLAST identification. The protein has a 99% similarity to the Human Fibroblast Binding Protein 2 (Ogawa et al., 2001). As such the FGFBP2 is commonly used as a synonym for the Killer-specific secretory protein 37. The FGFBP2 gene is conserved in chimpanzee, Rhesus monkey, chicken, zebrafish and frog. 137 Organisms have orthologs with human gene FGFBP2.

At the time of this invention no adequate studies on the levels of Ksp37 in LTNPs, or on the difference in levels of Ksp37 between LTNPs, HIV negative individuals, HIV positive individuals on HAART and HIV positive individuals who had not yet begun treatment, had been conducted.

In light of the above, there is a need to identify and characterise the proteins or peptides secreted by activated CD8⁺ T Lymphocytes of Long Term Non Progressors, to determine the levels of Ksp37 produced by these Long Term Non Progressors, and to utilise these results in the development of a therapeutic vaccine for the treatment or prevention of viral infection and/or viral cancers.

Embodiments of the invention thus aim to address the issues identified above, at least to some extent.

### SUMMARY OF THE INVENTION

The present invention concerns compositions and methods useful for the treatment of disease characterised by viral infection and/or diseases associated with immune system disorders and/or viral cancers in mammals, including humans.

The invention is based on identifying the optimum concentration range within which a protein identified as Ksp37, having a molecular weight in the range ranging from 24 kDa-45 kDa, enhances the immune response of a subject against viral infection and/or diseases associated with immune system disorders and/or viral cancers, and utilising this identified concentration range of the protein for the treatment of viral infection and/or diseases associated with immune system disorders and/or viral cancers as well as in the preparation of medicines and medicaments for the treatment of viral infection and/or diseases associated with immune system disorders and/or viral cancers.

According to the invention, the optimum concentration range within which the Ksp37 protein enhances the immune response of the subject against viral infection and/or diseases associated with immune system disorders and/or viral cancers, is 400 ng Ksp37 per mL of blood plasma-700 ng Ksp37 per mL of blood plasma.

The invention therefore provides for a method of protecting a subject from disease characterised by viral infection and/or diseases associated with immune system disorder and viral cancers, and or treating a subject suffering from disease characterised by viral infection and/or diseases associated with immune system disorder and viral cancers by raising the levels of Ksp37 in the blood plasma of the subject to a therapeutically effective concentration level, wherein the therapeutically effective level of Ksp37 is between 400 ng/mL-700 ng/mL.

The levels of Ksp37 in a subject may be increased by one or more of the following routes:
a) by administering a medicament comprising a therapeutically effective amount of a clinically modified or genetically engineered Ksp37 protein and/or proteins having a molecular weight in the range ranging from 24 kDa-45 kDa, to the subject; and/or
b) by stimulating the production of Ksp37 in the subject to a therapeutically effective level by administering a vector encoded with a KSP37 gene which will translate to a Ksp37 protein and/or proteins having a molecular weight in the range ranging from 24 kDa-45 kDa that is useful in fighting viral infection and/or diseases associated with immune system disorders and/or viral cancers to the subject; and/or
c) by stimulating the production of Ksp37 in the subject to a therapeutically effective level by chemically treating the subject with a polar compound, to activate increased Ksp37 production.

The therapeutically effective level of Ksp37 is preferably a blood plasma concentration level of 400 ng/mL-700 ng/mL, and a therapeutically effective amount of clinically modified or genetically engineered Ksp37 protein and/or proteins having a molecular weight in the range ranging from 24 kDa to 45 kDa is an amount that results in a Ksp37 blood plasma concentration level of 400 ng/mL-700 ng/mL.

The invention also provides a clinically modified or genetically engineered Ksp37 protein and/or proteins having a molecular weight in the range ranging from 24 kDa to 45 kDa, and/or a vector encoded with a KSP37 gene which will translate to a Ksp37 protein and/or proteins having a molecular weight in the range ranging from 24 kDa to 45 kDa for use in a method of protecting a subject from disease characterised by viral infection and/or diseases associated with immune system disorder and viral cancers.

The invention further provides for the use of a clinically modified or genetically engineered Ksp37 protein and/or proteins having a molecular weight in the range ranging from 24 kDa to 45 kDa, and/or a vector encoded with a KSP37 gene which will translate to a Ksp37 protein and/or proteins having a molecular weight in the range ranging from 24 kDa to 45 kDa, in the manufacture of a medicament for the treatment and/or protection of a subject from disease characterised by viral infection and/or diseases associated with immune system disorder and viral cancers, wherein the medicament increases the levels of Ksp37 protein in the subject to between 400 ng/mL and 700 ng/mL.

The invention yet further provides a pharmaceutical composition for use in a method of protecting a subject from disease characterised by viral infection and/or diseases associated with immune system disorder and viral, comprising a therapeutically effective amount of one or more of; a clinically modified or genetically engineered Ksp37 protein and/or proteins having a molecular weight in the range ranging from 24 kDa to 45 kDa, and/or a vector encoded with a KSP37 gene which will translate to a Ksp37 protein and/or proteins having a molecular weight in the range ranging from 24 kDa to 45 kDa, and/or a polar compound.

The therapeutically effective amount of Ksp37 protein or modified proteins, comprises an amount capable of increasing the levels of Ksp37 protein in the subject to between 400 ng/mL and 700 ng/mL when administered one or more times over a suitable period and is between 0.001 μg/kg body weight of a mammal microgram and 20 μg/kg body weight of a mammal.

The pharmaceutical composition or medicament may additionally include a pharmaceutically accepted excipient, including but not limited to water, saline, phosphate buffered solution, ringer's solution, dextrose solution, Hank's solution, polyethylene glycol containing physiological balanced salt solution and other aqueous physiologically balanced salt solution as well as non-aqueous vehicles, such as fixed oils, sesame seed oil, ethylene oleate triglycerides.

The pharmaceutical composition or medicament may also include a controlled release composition that is capable of slowly releasing Ksp37 into a mammal.

The pharmaceutical composition or medicament may be administered to a subject via an acceptable administration route, including nasal, oral, topical, inhalation, transdermal, rectal or parenteral administration.

Additional compounds capable of enhancing the ability of Ksp37 to protect a mammal from disease characterised by viral infection, may be included in the pharmaceutical composition or medicament, the compounds including, but not limited to compounds capable of regulating cell mediated immune response, regulating T-helper cell activity, regulating degranulation of mast cells, protecting sensory nerve endings, regulating eosinophil and or blast cell activity, and/or preventing or relaxing smooth muscle contractions.

The Ksp37 protein may be extracted from blood components and/or tissue then purified, acetylised, genetically engineered, cloned and transferred back to a mammalian host as a therapeutic and/or preventative vaccine against viral infection and/or diseases associated with immune system disorders and/or viral cancers.

The vector encoded with the KSP37 gene that is useful in fighting viral infection and/or diseases associated with immune system disorders and/or viral cancers, contains a nucleic acid sequence that translates to a protein identical to the naturally occurring Ksp37 protein.

An appropriate vector includes a pGEM-T Vector or a pCMV3-C-GFPSpark.

The hosts cells may include all blood components and mammalian tissue cells related to the hosts immune system which is identified as the primary location for production of Ksp37.

The invention yet further provides a pharmaceutical composition comprising a therapeutically effective amount of a Polar compound wherein the therapeutically effective amount of the polar compound is an amount sufficient to activate increased Ksp37 production to levels of between 400 ng/mL and 700 ng/mL in a subject.

The polar compound is preferably N,N-dimethylformamide (DMF).

A therapeutically effective dose of DMF for activating Ksp37 production may be a dose that results in a peak plasma level of about 2 mg/l-200 mg/l, more preferably about 100 mg/l-200 mg/l, still more preferably about 150 mg/l of DMF. Especially preferred is a peak plasma level of 100 mg/l-150 mg/l or 150 mg/l-200 mg/l of DMF.

The virus may be a retrovirus, and the viral cancer may include ovarian cancer, Leukaemia, Burkitt's lymphoma, nasopharyngeal carcinoma, and some forms of Hodgkin's disease.

The subject is a mammal.

The invention also provides a therapeutic and/or preventative vaccine against viral infection and/or diseases associated with immune system disorders and/or viral cancers, the vaccine comprising a therapeutically effective amount of a clinically modified or genetically engineered Ksp37 protein and/or proteins having a molecular weight in the range ranging from 24 kDa to 45 kDa, and/or a vector encoded with a KSP37 gene that is useful in fighting viral infection and/or diseases associated with immune system disorders and/or viral cancers.

### BRIEF DESCRIPTION OF THE DRAWINGS

FIG. 1 shows the real time PCT results confirming the HPLC peaks.

FIG. 2 shows a map of the pCMV3-C-DDK (Flag) expression vector.

### DETAILED DESCRIPTION OF THE INVENTION

The following description is provided as an enabling teaching of the invention, is illustrative of principles associated with the invention and is not intended to limit the scope of the invention. Changes may be made to the embodiment/s depicted and described, while still attaining results of the present invention and/or without departing from the scope of the invention. Furthermore, it will be understood that some results or advantages of the present invention may be attained by selecting some of the features of the present invention without utilising other features. Accordingly, those skilled in the art will recognise that modifications and adaptations to the present invention may be possible and may even be desirable in certain circumstances, and may form part of the present invention.

1. Comparing the Levels of Ksp37 Amongst Different Sample Groups

A sample of five (5) known LTNP individuals (LTNP1-LTNP5) as well as a random sample of fifteen volunteers were selected from the George Mukhari Academic Hospital. The fifteen random volunteers were further divided into the following classes:
5 volunteers who are HIV Positive and on HAART (HAART1-HAART5);
5 volunteers who have just been diagnosed with HIV and have not yet started with HAART (NAIVE1-NAIVE5); and
5 volunteers who are HIV negative (NEG1-NEG5).

The sample size of five (5) LTNP individuals was determined by the scarcity of known LTNP individuals.

Blood samples were drawn from these 20 individuals.

a. Cell Isolation

Isolation of total RNA from whole blood was done using an RNA extraction kit (Thermo Fischer Tempus Spin RNA Isolation Kit), according to the manufacturer's instructions. Cells were separated from whole blood by centrifugation at 250×g for 5 minute/s (min/s). Next the cell pellets were washed twice with ice cold PBS, pH7.4, resuspended in 600 μl lysis buffer supplemented with s-mercaptoethanol, followed by vortex mixing for 10 second/s (sec/s). 360 μl of ethanol were added to cell lysate, and mixed by aspiration. The 700 μl lysate was then transferred onto the RNA purification column inserted in a collection tube. The column contents were subjected to centrifugation for at 12 000×g for 1 minute. The flow-through was discarded. The purification column was washed with wash buffers 1 and 2, followed by centrifugation at 12 000×g for 1 minute, with the flow-through discarded. RNA was eluted with 50 μl nuclease-free sterile deionized water, followed by centrifugation at 12 000×g for 1 minute. The elute RNA was stored at −70° C.

b. Conversion of RNA to cDNA

Extracted RNA was reverse transcribed to cDNA using the cDNA synthesis kit (Thermo Scientific Superscript VILO cDNA Synthesis Kit), according to the manufacturer's instructions. The cDNA reaction mixture contained 10 μl template RNA, 2 μl oligo d(T) primer, 12 μl nuclease-free deionized water, 4 μl 5× reaction buffer, 1 μl RibobLock RNase inhibitor and 1 μl MuLV reverse transcriptase. The resulting cDNA was stored at −70° C. until used.

c. Amplification and Quantification of the Resultant cDNA

The cDNA was amplified using polymerase chain reaction kit (Kapa Bio systems, USA) according to the manufacturer's instructions. The PCR reaction mixture contained 2 μl forward 5′-CTTCCGAGGGTGACAGGTGA-3′ and reverse 5′-TCCAGTGTGAGAACGTTGGATTG-3′ primers (0.4 μM each), 5 μl of template cDNA, 16 μl of nuclease free deionized water and 25 μl of 2× Ready Mix. The PCR reaction consisted of 90 s denaturation at 9513, primer annealing for 30 s at 59° C. and elongation for 1 min at 72° C. for 7 min. PCR products were analyzed on 2 percent (%) agarose gel electrophoresis at 75 Volts (V) for at least 60 mins at room temperature. The gel was viewed, and band intensities estimated using Chemo (Bio-Rad). Protein polymorphism was then determined using restriction fragment length polymorphism.

d. Cytokines Quantification in Different Groups

The quantities of selected cytokines (IFN-γ, IL-5, GM-CSF, TNF-aIL-2, IL-13, IL-41L-10, IL12p70) were determined amongst the different groups using direct, quantitative measurement of cytokine proteins in single human CD8 lymphocytes from fresh peripheral blood of healthy donors (Saxena et al., 2018 Ultrasensitive Quantification of Cytokine Proteins in Single lymphocytes from Human Blood following ex-vivo stimulation Front. Immunol., 9:2462. doi: 10.3389/fimmu.2018.02462.eCollection2018).

Results

Real time PCR analysis showed Ksp37 levels as indicated in the table below:

TABLE 1
Ksp37 Values

| Sample | Ksp37 value (ng/ml) |
|---|---|
| LTNP1 | 507.24 |
| LTNP2 | 473.23 |
| LTNP3 | 530.99 |
| LTNP4 | 571.53 |
| LTNP5 | 655.88 |
| HAART1 | 173.98 |
| HAART2 | 152.70 |
| HAART3 | 158.31 |
| HAART4 | 158.90 |
| HAART5 | 176.70 |
| NAIVE1 | 375.34 |
| NAIVE2 | none |
| NAIVE3 | 348.40 |
| NAÏVE4 | 236.28 |
| NAIVE5 | 433.81 |
| NEG1 | 243.49 |
| NEG2 | 274.41 |
| NEG3 | 249.38 |
| NEG4 | none |
| NEG5 | none |

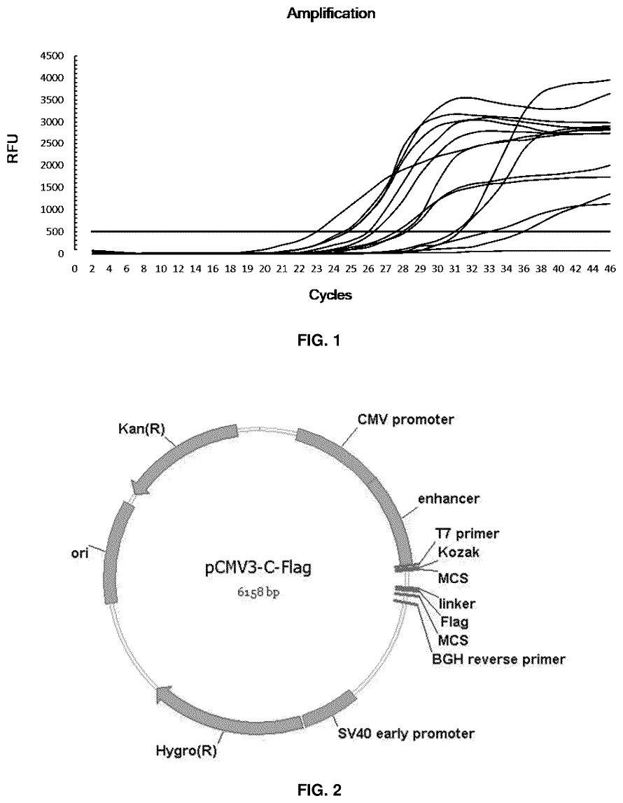

The real-time PCR results confirm the HPLC peaks observed by the fewer cycles for detection of Ksp37 in LTNP blood serum (average 26 cycles) when compared to the serum of other study groups as reflected in FIG. 1.

The results indicate that the blood serum of Long-term Non-Progressor Group had significantly higher levels of the protein Ksp37.

The protein was also seen to be associated with higher levels of other cytokines and proteins namely IL-12p70, IFN-γ and IL-4 as illustrated in the following table 2. Levels are measured in ng/mL

TABLE 2
Cytokine Results

| Sample | IFN-γ | IL-5 | GM-CSF | TNF-a | IL-2 | IL-13 | IL-4 | IL-10 | IL12p70 |
|---|---|---|---|---|---|---|---|---|---|
| LTNP1 | 4.5 | 3.5 | −1 | 15 | 4 | 6.5 | 3.5 | 1.5 | 4 |
| LTNP2 | 2.5 | 2 | −2 | 8.5 | 4 | 3 | 2 | 5.5 | 2.5 |
| LTNP3 | 2 | 3 | −1 | 45.5 | 4 | 2.5 | 1.75 | 4 | 2.5 |
| LTNP4 | 2.5 | 3.25 | 1.5 | 85 | 8.5 | 3 | 2.5 | 0 | 2.5 |
| LTNP5 | 3.5 | 2.5 | 1 | 105 | 7.75 | 1 | 1 | −10 | 2 |
| HAART1 | 0 | 0.5 | −5.5 | 12 | 1 | 0 | 0.5 | 0 | 0 |
| HAART2 | 0 | 2.5 | −3.5 | 26 | 4 | 2 | 2.5 | 6 | 1 |
| HAART3 | −1 | 0.5 | −4.5 | 16 | 1 | 1 | 0.5 | −1 | 0 |
| HAART4 | 2 | 0.5 | −4.5 | 6 | 0 | 2 | 0.5 | 1 | 1 |
| HAART5 | 6 | 1.5 | −2.5 | 10 | 0.5 | 2 | 0.5 | 2.5 | 2 |
| NAIVE1 | −1 | −0.5 | −6.5 | 18 | 0 | 1 | 0.5 | −9 | 0 |
| NAIVE2 | 0 | 2.5 | −2.5 | 6 | 0 | 1 | −0.5 | −2 | 1 |
| NAIVE3 | 0 | 0.5 | −5.5 | 10 | 0 | 1 | −0.5 | 1 | 0 |
| NAÏVE4 | 1 | 1.5 | −4.5 | 3 | 1 | 1 | −0.5 | 1 | 1 |
| NAIVE5 | 0 | 1.0 | −4.0 | 6 | 0 | 1 | 0 | 1 | 0 |
| NEG1 | 0 | 0.5 | −5.5 | 5 | 0 | 0 | −0.5 | −2 | 0 |
| NEG2 | 1 | 0.5 | −2.5 | 8 | 1 | 1 | 0.5 | 1 | 0 |
| NEG3 | 0 | 1.5 | −5.5 | 6 | 0 | 2 | 0.5 | 4 | 1.5 |
| NEG4 | 1 | 2.5 | −4.5 | 19 | 0.5 | 3 | 0.5 | 0.5 | 0 |
| NEG5 | 1 | 1.5 | −5.5 | 11 | 1 | 1 | 0.5 | 0 | 1 |

While it should be borne in mind that the samples used were from cultured cells and the values tabled above are therefore not representative of cytokines values that would be expected from samples taken directly from a human host, the values do, however, guide the observations as to the pathways of immunity associated with high levels of Ksp37.

The production of IFN-γ and IP-12p70 is partly regulated by a positive feedback loop, with IFN-γ and GM-CSF promoting IL-12p70 production and IL-12p70 in turn stimulating IFN-γ and GM-CSF secretion. IL-12p70 and IFN-γ promote Th1 differentiation, favouring cell-mediated immunity and inhibiting Th2 responses.

When considering the above IFN-γ results, the average value in LTNPs is considerably higher than the values from the other study groups. IFN-γ is a cytokine critical to both innate and adaptive immunity, and functions as the primary activator of macrophages, in addition to stimulating natural killer cells and neutrophils.

IFN-γ has been identified as a correlator of better disease prognosis in HIV infection, and is positively associated with CD8+ T cell and activated NK cell counts (López M et al., 2011‘The expansion ability but not the quality of HIV-specific CD8+ T cells is associated with protective human leucocyte antigen class I alleles in long-term non-progressors’, Immunology, 134(3), pp. 305-313. doi: 10.1111/j.1365-2567.2011.03490.x.).

Similarly, when considering the IL-12p70 values, the average value in LTNPs is considerably higher than the values from the other study groups. IL-12p70 stimulates growth and function of T cells, production of interferon-gamma (IFN-γ) and tumour necrosis factor-alpha (TNF-α) from T cells and natural killer (NK) cells, and reduces IL-4-mediated suppression of IFN-γ.

In SIV-infected macaques, IL-12p70 treatment during acute infection was associated with decreased viral loads, increased CD8⁺ NK and T cells, reduced naïve CD4+ T cells expressing homing markers, retention of HIV-specific CTL and prolonged survival.

The above results also point to higher levels of IL-4 in the LTNP group when compared to other study groups. IL-4 has many biological roles, including the stimulation of activated B-cell and T-cell proliferation, and the differentiation of B cells into plasma cells. It is a key regulator in humoral and adaptive immunity. IL-4 induces B-cell class switching to IgE, and up-regulates MHC class II production. IL-4 and IL-12p70 have a complementary role. The main function of the IL-4 is to stimulate the adaptive immune system and CD8⁺ cytotoxic cells. Whole IL-12p70 prevents the suppression of IL-4.

A recent study also indicated that IL-10 can significantly inhibit HIV-1 replication in monocytes/macrophages. The inhibitory effects of IL-10 on HIV-1 production in monocytes/macrophages are the result of IL-10-induced inhibition of the synthesis of other cytokines, such as tumour necrosis factor alpha and IL-6, capable of up-regulating HIV-1 expression in these cells.

2. Modes of Action of Ksp37

While research into the modes of action of Ksp37 is ongoing, increased levels of Ksp37 appear to inhibit the progression of HIV into AIDS by a number of different mechanisms. These include:

a. CD8+ Consideration

One possible mechanism by which increased levels of Ksp37 inhibit the progression of HIV to AIDS is linked to the site of production of Ksp37, the HIV GAG-specific cells. The GAG-specific CD8⁺ cells are characterised by the production levels of CD107a IFN-γ MIP-1β IL-2 TNF-α (T, H. C. D. et al. (2006) ‘IMMUNOBIOLOGY HIV nonprogressors preferentially maintain highly functional’, Blood, 107(12), pp. 4781-4789. doi: 10.1182/blood-2005-12-4818).

The production of these components are believed to negate the immunosuppressive abilities of HIV. Ksp37 is responsible for increasing the life span of these HIV specific CD8⁺ cells. Ksp37 is hypothesised to control the immunosuppressive effects of HIV by also controlling the release of components such as perforin, TNF-α, and IL-2 from the CD8⁺ cells. The initial response to HIV infection in LTNP individuals is the same as seen in other study groups. Once the HIV specific CD8⁺ cells have been activated in the LTNP individuals, a few outcomes are observed. The levels of TNF-α rise but decrease as the levels of Ksp37 increase, as shown in Table 3 below:

TABLE 3
Levels of TNF-α and Ksp37

| | Initial Ksp37 | Ksp37 levels | | TNF-α |
|---|---|---|---|---|
| | levels before | 4 hrs after HIV | Initial TNF-α | levels |
| | HIV infection | infection. | levels | after 4 hrs |
| LTNPs | 441 ± 235 | 800 ± 529 | 9 ± 2 pg/mL | 3 ± 1 pg/mL |
| | ng/ml | ng/ml | | |
| Progressors | 380 ± 200 | 246 ± 127 | 7 ± 2 pg/mL | 15 ± 6 |
| | ng/ml | ng/ml | | pg/mL |

The main role of TNF-α in the body is linked to inflammation in the human body. The decreased levels of TNF-α decreases inflammation at the site of activation, thus lowers response from the CD4⁺ cells. This leaves the virus exposed in the blood stream and unable to infect the CD4⁺ host cells. This allows for the HIV specific CD8⁺ cells to directly attack the virus. Viruses are typically eliminated by virus-specific CD8⁺ T cells, which recognize processed viral proteins that are presented as a complex with an HLA class I molecule at the surface of an infected cell.

Recognition through the T cell receptor (TCR) initiates a cascade of activation events, ultimately leading to the release of granzymes and perforin and killing of the infected cell, which can occur before infectious progeny virions are produced (R. Brad Jones et al., 2016).

High IL-2 levels lead to a decrease in overall generation of early memory T cells by both decreasing central memory T cells and augmenting effectors (T. Kaartinen et al., 2017). There is, therefore, an inverse relationship between the levels of IL-2 and the generation and/or lifespan of CD8⁺ T cells. Ksp37-expressing CD4 and CD8 T cells lack the ability to produce IL-2. (Ogawa et al., 2001). Therefore, high levels of Ksp37 are associated with lower levels of IL-2 production at activation. The levels of Ksp37 are therefore inversely proportional to the levels of IL-2.

Therefore, when Ksp37 levels are high, IL-2 is low, and CD8⁺ memory cell generation increases with a longer lifespan. This supports a greater ability to kill the virus. The initial activation of the immune system is as a result of IL-2, and once IL-2 is removed this results in massive infected cell death. As HIV-specific CD8+ cells lack the ability to produce IL-2 (Ogawa et al., 2001), these are not heavily affected by IL-2 levels.

As noted, the sample study utilised cells grown inside the lab, and outside of the hosts immune system, as a result the IL-2 levels recorded are not fully representative of what would be normally found in LTNP hosts.

The action of polar agents on oncogenic expression is hypothesized to be the induction of cancer cells to become more benign. It would seem reasonable that to convert a malignant cell to a benign type, there should be some modulation of gene expression causing malignancy in the first place.

A study done by Ogawa et al in 2001 suggest that Ksp37 may be involved in an essential process of cytotoxic lymphocyte-mediated immunity in patients with Epstein-Barr virus and that Ksp37 may also have clinical value as a new type of serum indicator for monitoring cytotoxic lymphocytes in vivo. EBV is associated with Burkitt's lymphoma, nasopharyngeal carcinoma, and some forms of Hodgkin's disease. EBV can readily infect and alter the genetic code of human B cells, and may predispose immunosuppressed patients to malignant tumours.

b. Ksp37 as a Vif Inhibitor

Ksp37 is also believed to act as a Vif inhibitor.

The body's natural immune response to retroviruses is based on the function of the APOBEC3 proteins. Human APOBEC3 (A3) proteins are cellular cytidine deaminases that potently restrict the replication of retroviruses by hypermutating viral cDNA and/or inhibiting reverse transcription. There are seven members of this family including A3A, B, C, D, E, F, G, and H, all encoded in a tandem array on human chromosome 22. A3F and A3G are the most potent inhibitors of HIV-1, but only in the absence of the virus-encoded protein, Vif (Shingo K, et al., 2011).

The cytidine deaminase APOBEC3G (A3G) exerts a multifaceted antiviral effect against HIV-1 infection. Firstly, A3G was shown to be able to terminate HIV infection by deaminating the cytosine residues to uracil in the minus strand of the viral DNA during reverse transcription (Sadler H et al., 2010 ‘APOBEC3G Contributes to HIV-1 Variation through Sublethal Mutagenesis’, Journal of Virology, 84(14), pp. 7396-7404. doi: 10.1128/jvi.00056-10.). A number of studies have also indicated that A3G inhibits HIV-1 reverse transcription by a non-editing-mediated mechanism.

HIV Vif antagonizes the human antiviral protein APOBEC3G by hijacking the human Elongin B/C (EloBC)-cullin-SOCS box (ECS)-type E3 ubiquitin ligase, resulting in the polyubiquitination of APOBEC3G and subsequently its proteasomal degradation (Matsui Y et al., 2016 ‘Core binding factor β protects HIV, type 1 accessory protein viral infectivity factor from MDM2-mediated degradation’, Journal of Biological Chemistry, 291(48), pp. 24892-24899. doi: 10.1074fjbc.M116.734673.).

HIV Vif protein has a similar function to the p23 antigen found on the Epstein Barr Virus (ËBV”) (among other functions, in their respective interactions with the Hsp90/70 chaperone proteins). The antigen p23 is an Isoform of the Vif HIV protein. A protein isoform, or “protein variant”, is a member of a set of highly similar proteins that originate from a single gene or gene family and are the result of genetic differences. Protein Isoforms tend to have the same or similar biological functions. Ogawa et al. found that in the presence of EBV there was a greater production of Ksp37. This indicates the antiviral properties of Ksp37.

It is believed that the mechanism of Ksp37 in supporting the function of APOBEC3G proteins in fighting EBV is similar to the mechanism of Ksp37 in supporting the function of APOBEC3G proteins in fighting HIV, given the similarity of the HIV Vif and EBV p23 antigen.

Therefore, the mechanism of action for Ksp37 may give it a sub-classification of a Vif inhibitor, inhibiting the formation of the E3 ligase complex. The possible inhibition of the Vif protein would allow the A3G protein to disrupt the translation of viral DNA.

3. Concentration Levels at which Ksp37 is Most Effective

The results in Tables 1 to 3 above indicate that Ksp37 is functional in blood serum concentration levels of between 400 ng/mL and 700 ng/mL.

Ksp37 is present in normal healthy individuals at concentrations lower than 400 ng/mL. At this level it is not effective in inhibiting the immune disabling replication of the HIV virus and possibly other retroviruses. Conversely, concentration levels of Ksp37 higher than 750 ng/mL may lead to autoimmune disorders, including asthma and Down Syndrome, due to the positive effect Ksp37 has on cytokines such as IL-5 and TNF-α which are associated with the increased activity of the immune system.

In light of this, the invention provides for a method of protecting a subject from disease characterised by viral infection and/or diseases associated with immune system disorder and viral cancers, by increasing the levels of Ksp37 in the subject to between 400 ng/mL and 700 ng/ml.

The levels of Ksp37 in a subject may be increased by one or more of the following routes:
(a) by administering a therapeutically effective amount of a clinically modified or genetically engineered Ksp37 protein and/or proteins having a molecular weight in the range ranging from 24 kDa to 45 kDa, to the subject;
(b) by stimulating the production of Ksp37 in the subject to a concentration level of 400 ng/mL-700 ng/mL by administering a vector encoded with KSP37 gene which will translate to a Ksp37 protein to the subject; or
(c) by stimulating the production of Ksp37 in the subject to a concentration level of 400-700 ng/mL by treating the subject with a polar compound, to activate increased Ksp37 production.

Subjects may include all mammals, and are not restricted to humans only.

4. Administering a Clinically Modified or Genetically Engineered Ksp37 Protein to a Subject

In one embodiment of the invention, a Ksp37 protein and or proteins in the range ranging from 24 kDa to 45 kDa which has been clinically modified/cloned or genetically engineered, and/or a formulation comprising a Ksp37 protein and or proteins in the range ranging from 24 kDa to 45 kDa which has been clinically modified/cloned or genetically engineered, may be administered to a subject to treat or prevent disease characterised by a viral infection and/or diseases associated with immune system disorder and viral cancers.

The subject is a mammal.

The protein is selected from the group of Ksp proteins and all it species, where said protein is a mammalian protein.

The protein is has a molecular weight of approximately 37 kDa with 223 amino acid chains, contains an N-terminal signal sequence, a short C-terminal hydrophobic region, and a potential O-Glycosylation site and a cysteine side chain.

The protein may be extracted from blood components and/or tissue then purified, acetylised, genetically engineered, cloned and transferred back to a mammalian host as a therapeutic and/or preventative vaccine for protection against or treatment of viral infections and/or diseases associated with immune system disorder and viral cancers.

One or more recombinant molecules can be used to produce the Ksp37 protein ex vivo. In one embodiment, an encoded product is produced by expressing a nucleic acid molecule under conditions effective to produce the protein.

A preferred method to produce an encoded protein involves transfecting a host cell with one or more recombinant molecules having a nucleic acid sequence encoding a Ksp37 protein, to form a recombinant cell. Suitable cells for transfection are any cells that can be transfected. Host cells can either be transfected cells or cells that are already transformed with at least one nucleic acid molecule.

Host cells useful in the present invention can be any cell capable of producing a Ksp37 protein, including bacterial, fungal, mammal and insect cell.

Transfection of a nucleic acid molecule into a host cell can be accomplished by any method by which a nucleic acid molecule can be inserted into a cell. Transfection techniques include but are not limited to, transfection, electrophoretic, microinjection, lipofection, adsorption and protoplasm fusion. In the use of recombinant DNA technology, expression can be improved by transfected nucleic acid molecules within a host cell, the efficiency with which nucleic acid molecules encoding a Ksp37 can be transcribed, the efficiency with which the resultant transcripts are translated and efficiency of post translational modification recombinant techniques useful for increasing expression of nucleic acid molecules.

Ex vivo production of Ksp37 protein includes, but is not limited to, operatively linking nucleic acid molecules to high-copy number plasmids, integration of the nucleic acid molecules into one or more host cells chromosomes, addition of vector stability sequences to plasmids, sub sectioning or modification of transcription control signals (promotors, operators, enhancers) substitutions or modifications of translational control signals (e.g. ribosome binding sites, Shere-Dalogans Signals), modifications of nucleic acid molecules to correspond to the codum usage of the host cells and deletion of sequences that destabilise transcripts.. The activity of an expressed recombinant Ksp37 protein maybe improved by fragmenting, modifying or derivatising nucleic acid molecules encoding such a protein.

Delivering a Formulation, Including a Ksp37 Protein to a Target Cell in a Mammal.

A “Target Site” refers to a site in a mammal to which one desires to deliver a therapeutic formulation. For example, a target site can be a lymphocyte, stem cells, all blood components, and other delivery vehicles including, but not limited to, natural lipid-containing delivery vehicles, including cells and cellular membranes; and artificial lipid-containing delivery vehicles including liposomes and micelles.

The delivery vehicle can be modified by known techniques to target a specific site in a mammal, thereby targeting and making use of a nucleic acid molecule at that site.

Suitable modifications include manipulating the chemical formula of the lipid position of the delivery vehicle and/or introducing into the vehicle a compound capable pf specifically targeting a delivery vehicle to a preferred site, for example, a preferred cell type. Specially targeting refers to causing a delivery vehicle to bind to particular cell by the interaction of the compound in the vehicle to a molecule on the surface of the cell. Suitable targeting compounds include ligands capable of selectively binding another molecule at a particular site. Example of such ligands includes antibodies, antigens, receptors and receptor ligands.

Manipulating the chemical formula of the lipid position of the delivery vehicle can modulate the extra cellular or intracellular targeting of the delivery vehicle. For example, a chemical can be added to the lipid formula of a liposome that alters the charge of the lipid bi layer of the liposome so that the liposome fuses with particular cells having a particular charge characteristics.

Excipients

A formulation comprising the Ksp37 protein to be administered to a subject can also include other components such as a pharmaceutically acceptable excipient. For example, formulations of the present invention can be formulated in a excipient that the subject can tolerate, examples of such excipients include water, saline, phosphate buffered solution, ringers solution, dextrose solution, Hank's solution, polyethylene glycol containing physiological balanced salt solution and other aqueous physiologically balanced salt solution. Non-aqueous vehicles, such as fixed oils, sesame seed oil, and ethylene oleate triglycerides can also be used.

Other useful formulations include suspensions containing viscosity enhancing agents such as sodium carboxymethyl cellulose, sorbitol, or dextrin. Excipients can also contain minor amounts of additives, such substances that enhance isotonicity and chemical stability or buffers. Examples of buffers include phosphate buffer, bicarbonate buffer, tres buffer, while examples of preservatives include Trimeresal, m-oro-cresol, formalin and benzyl alcohol.

Standard formulation can be either liquids or injectable or solids which can be taken up in a suitable liquid as a suspension or solution for injection. Thus, in a non-liquid formulation, the excipient can comprise of dextryls, Human serum albumin, preservatives etc. to which sterile water or saline can be added prior to administration.

The Ksp37 protein maybe administered by at least one route elected from the group consisting of oral, nasal, topical, by inhalation, transdermal, rectal and parenteral (subcutaneous/intramuscular) administration.

Controlled Release

A formulation including the Ksp37 protein or modified protein to be administered to a mammal may include a controlled release composition that is capable of slowly releasing Ksp37 into a mammal. As used herein a controlled release composition comprises a Ksp37 protein or in a controlled release vehicle.

Suitable controlled release vehicles include but are not limited to biocompatible polymers, other polymeric matrix capsules, micro capsules, micro particles, Bolus preparations, Osmotic pumps, diffusion devices, liposomes, lipospheres, dry powders and transdermal delivery systems. Other controlled release of the invention includes liquids that, upon administration to a mammal form a solid or gel in-situ.

Additional Compounds

Additional compounds capable of enhancing the ability of Ksp37 to protect a mammal from disease characterised by viral infection may be administered sequentially or simultaneously. Such compounds include compounds capable of regulating cell mediated immune response, regulating T-helper cell activity, regulating degranulation of mast cells, protecting sensory nerve endings, regulating eosinophil and or blast cell activity, and/or preventing or relaxing smooth muscle contractions. Such compounds will further induce microvascular permeability or modulating Th1 and or Th2 cell subset differentiation.

The choice of compound to be administered in conjunction with Ksp37 protein can be made by one of skill in the art based on various characteristics of the mammal. In particular a mammal's genetic background, health history, physical signs, use of rescue medication and blood gases, and blood analysis.

Dose

A therapeutic dose of Ksp37 protein or modified proteins administered to a mammal, comprises a dose capable of protecting a mammal from and/or treating a disease characterised by infections and/or a Th-1 type immune response, when administered one or more times over a suitable period.

Alternatively, a therapeutic dose of Ksp37 protein or modified proteins comprises a dose that improves the immune system of a mammal. Further alternatively, a therapeutic dose of Ksp37 protein or modified protein comprises a dose that reduces viral infections and/or increases Th 1-type cytokines.

A preferred single dose pf Ksp37 protein or modified proteins, which is hypothesised to produce a therapeutic or preventative result has been identified to be of between 0.001 μg/kg body weight of a mammal microgram and 20 μg/kg body weight of a mammal.

4. Method of Stimulating Expression of KSP37 in a Cell

Gene therapy is a new therapeutic modality under consideration for the treatment of various inherited and acquired disorders. It works on the premise of manipulating gene expression towards a therapeutic end. Recent advances in biotechnology have stimulated the development of in vivo gene therapy approaches based on the direct delivery of the therapeutic gene to the cells in vivo. Gene therapy aims to introduce a normal copy of the gene in question to restore, increase, or to modify the function of a protein.

A Nucleic acid molecule encoding a Ksp37 protein can be obtained from its natural source, either as an entire (complete) gene or a portion thereof. Alternatively, a nucleic acid molecule can be produced using recombinant DNA technology (Polymerase Chain Reaction amplification cloning) or chemical synthesis.

Nucleic acid molecules include natural nucleic acid molecules and homologues thereof including but not limited to, natural allelic variants and modified nucleic acid molecules in which nucleotides have been inserted, deleted substituted and/or inverted in such a manner that such modifications do not substantially interfere with nucleic acid ability to encode Ksp37 protein useful in the method of the present invention.

In one embodiment, a nucleic acid molecule encoding a Ksp37 protein that is useful in fighting viral immune system related infections such as HIV and viral cancers, is a nucleic acid sequence that translates to a protein that is identical to the naturally occurring Ksp37 protein.

An isolated, or biological pure nucleic acid molecule, is a nucleic acid molecule that has been removed from its natural milieu.

A nucleic acid molecule encoding a Ksp37 protein can be produced using any of a number of methods known to those skilled in the art, including recombinant DNA techniques, such as site directed mutagenesis, chemical treatment of nucleic acid molecules with polar compounds to induce mutation, restriction enzymes cleavage of a nucleic acid fragment, irrigation of nucleic acid fragments, polymerase chain reaction (PCR) and or mutagenesis of select regions of nucleic acid sequence, synthesis of Oligonucleotide mixtures and ligation of mixture groups to build a mixture of nucleic acid molecules and combinations thereof. Nucleic acid molecule envelopes can be selected from a mixture of modified nucleic acid by screening of the function encoded by the nucleic acid.

A nucleic acid molecule used for encoding a Ksp37 protein that is useful in the method of present invention can be operatively linked to one or more transcription control sequences to form a recombinant molecule. The phrase “operatively linked” refers to linking nucleic acid molecule to a transcription controlled sequence in a manner such that the molecule is able to be expressed when transfected, transduced or transformed into a whole cell. Transcription control sequences are sequences which control the initiation, elongation and termination of transcription. Of particular importance are controlled transcription initiation, promotor, enhancer, operator and repressor sequences.

Suitable transcription control sequences include any transcription control sequence that can transcript in a recombinant cell useful for the expression of the Ksp37 protein, and or useful to administer in a mammal in the method of the present invention, Preferred transcription control sequences include those that function in mammalian, bacterial, or insect cells.

Transcription controlled sequence of the present invention can also include naturally occurring transcriptions controlled sequences naturally associated with gene encoding a KSP 37 protein useful in a method of the present invention.

Recombinant molecules of the present invention, which can be either DNA or RNA, can also contain additional regulatory sequences, such as translation regulatory sequences, origins or replications, and other regulatory sequences that are compatible with the recombinant cell. In one embodiment, a recombinant molecule of the present invention contains secretory signals (signal segment nucleic acid sequences) to enable an expressed Ksp37 protein to be secreted from a cell that produces the protein.

Suitable signal segments include, but are not limited to, signal segments naturally associated with any of the here afore mentioned Ksp37 proteins and all associated species and nucleotides of Ksp37 proteins.

The rate limiting technologies of gene therapy are the gene delivery vehicles, known as vectors, used to accomplish gene transfer. Vectors can also be used to increase gene production of a specific protein.

Suitable Vector

Examples of pharma grade acceptable vectors which are particularly useful for the administration of nucleic acid molecules encoding Ksp37 protein are:
pGEM-T Vector
pCMV3-C-GFPSpark

These are some of the vectors that are currently used on the market as vectors for KSP37 sequence.

An expression vector, otherwise known as an expression construct, is usually a plasmid or virus designed for gene expression in cells. The vector is used to introduce a specific gene into a target cell, and can commandeer the cell's mechanism for protein synthesis to produce the protein encoded by the gene.

The gene of interest in this case is KSP37, which is normally produced by CD8⁺ cells part of the adaptive immunity in human hosts (López et al., 2011).

The pCMV3-C-DDK (Flag) is an expression vector (FIG. 2) that is used for the expression of KSP37 in mammalian cells. The vector should contain specific segments to allow expression, these include a promoter, the correct translation initiation sequence such as a ribosomal binding site and start codon, a termination codon, and a transcription termination sequence. After the expression of the gene product, it may be necessary to purify the expressed protein; however, separating the protein of interest from the great majority of proteins of the host cell can be a protracted process. To make this purification process easier, a purification tag may be added to the cloned gene.

Vectors are transfected into the cells and the DNA may be integrated into the genome by homologous recombination in the case of stable transfection, or the cells may be transiently transfected. Examples of mammalian expression vectors include the adenoviral vectors, the pSV and the pCMV series of plasmid vectors, vaccinia and retroviral vectors, as well as baculovirus. The promoters for cytomegalovirus (CMV) and SV40 are commonly used in mammalian expression vectors to drive gene expression.

In particular, pGEM-T vector, KSP37/FGFBP2 cDNA ORF CLONE supplied by SinoBiological has been identified as a suitable cloning vector of the full-length clone DNA of human fibroblast growth factor binding protein 2.2 units to 10 units, for use in gene therapy against retroviral infections, viral cancers and prions in mammals.

Excipients/Delivery Vehicles

According to the present invention, a nucleic acid molecule encoding a Ksp37 protein may be administered with a pharmaceutically accepted excipient. A pharmaceutically accepted excipient can include but is not limited to a natural lipid containing substrate, an oil, an ester, glycol, a virus, a metal particle or cationic molecule.

A pharmaceutical acceptable excipient which is capable of targeting is herein referred to as a “delivery vehicle”. Pharmaceutical acceptable excipients of the present invention are capable of delivering a formulation, including a Ksp37 protein and/or nucleic acid molecule encoding a Ksp37 protein, to a target cell in a mammal. A “Target Site” refers to a site in a mammal to which one desires to deliver a therapeutic formulation. For example, a target site can be a lymphocyte, stem cells, all blood components.

Delivery vehicles include, but are not limited to, natural lipid-containing delivery vehicles, including cells and cellular membranes, artificial lipid-containing delivery vehicles, including liposomes and micelles.

A delivery vehicle of the present invention can be modified to target a specific site in a mammal, thereby targeting and making use of a nucleic acid molecule at that site. Suitable modifications include manipulating the chemical formula of the lipid position of the delivery vehicle and/or introducing into the vehicle a compound capable of specifically targeting a delivery vehicle to a preferred site, for example, a preferred cell type.

Specially targeting refers to causing a delivery vehicle to bind to a particular cell by the interaction of the compound in the vehicle with a molecule on the surface of the cell. Suitable targeting compounds include ligands capable of selectively binding another molecule at a particular site. Examples of such ligands include antibodies, antigens, receptors, and receptor ligands.

Manipulating the chemical formula of the lipid position of the delivery vehicle can modulate the extra cellular or intracellular targeting of the delivery vehicle. For example, a chemical can be added to the lipid formula of a liposome that alters the charge of the lipid bilayer of the liposome so that the liposome fuses with particular cells having particular charge characteristics.

Administration of the Vector

The vector is administered to the subject via an acceptable administration route, including nasal, oral, topical, inhalation, transdermal or parenteral administration.

Additional Compounds

Additional compounds capable of enhancing the ability of Ksp37 to protect a mammal from disease characterised by viral infection may be administered sequentially or simultaneously. Such compounds include compounds capable of regulating cell mediated immune response, regulating T-helper cell activity, regulating degranulation of mast cells, protecting sensory nerve endings, regulating eosinophil and or blast cell activity, and/or preventing or relaxing smooth muscle contractions. Such compounds will further induce microvascular permeability or modulating Th1 and or Th2 cell subset differentiation.

The choice of compound to be administered in conjunction with a nucleic acid molecule encoding a Ksp37 protein can be made by one of skill in the art based on various characteristics of the mammal. In particular a mammal's genetic background, health history, physical signs, use of rescue medication and blood gases, and blood analysis.

7. Chemical Treatment with Polar Compounds to Activate Ksp37 Production

As previously disclosed, the polar compounds dimethylformamide (DMF) and DMSO have been shown to be powerful inhibitors of HIV production. Treatment of patients infected with HIV-1 with a transdermal patch containing dimethylformamide (DMF) showed promising results.

The action of polar agents on oncogenic expression is hypothesized to be the induction of cancer cells to become more benign. It would seem reasonable that to convert a malignant cell to a benign type, there should be some modulation of gene expression causing malignancy in the first place. In the HL-60 human promyelocytic leukaemia cells, Diemthylsulfoxide [DMSO] reduced the expression of c-myoncogenes by 80 to 90% [5].

It is implicated that chemical inducers of cell differentiation play an important role in viral replication by affecting cellular mechanisms of the host cell.

Polar compounds, including DMF may be used in the clinical management of viral infection and diseases through a cascade of events that starts with the activation of CD8 cells, which in turn activate production of Ksp37.

Polar compounds such as dimethylformamide [DMF] may be used as an activator of KSP37 protein.

DMF is generally used as a polar solvent and is readily absorbed through the skin, by inhalation, and upon oral ingestion. DMF is rapidly metabolized, mainly in the liver, and excretion occurs principally in the urine.

Transdermal and suppository delivery of chemicals is a common method of altering hormonal properties of a patient.

DMF may be administered to a patient via transdermal application, using any suitable drug delivery device, for example by applying one or more dermal patches, or a suppository for rectal application to activate Ksp37 production. Treatment with a dermal patch to activate Ksp37 production would comprise application of the patch to the skin for a period of about 8 hours once a week, while treatment by rectal application of DMF to activate Ksp37 production would ideally comprise use of a suppository once a week.

A therapeutically effective dose of DMF for activating Ksp37 production be a dose that results in a peak plasma level of about 2 mg/l-200 mg/l, preferably about 100 mg/l-200 mg/l, still more preferably about 150 mg/l of DMF. Especially preferred is a peak plasma level of 100 mg/l-150 mg/l or 150 mg/l-200 mg/l of DMF.

For transdermal administration of a polar compound, the rate of absorption is determined by the skin of the subject. Upon exposure to the human skin, liquid DMF is absorbed at a steady-state rate of approximately 9.4 mg/cm²/hour (see Mraz and Nohova, 1992, Occup. Env. Health 64:85-92).

Accordingly, the desired rate of absorption may be achieved by controlling the surface area of the skin exposed to the drug, as by determining the area of each patch and the number of patches applied to the skin. For example, two patches of diameter 9 cm will expose a total skin surface area of 127 cm² to the polar compound; for DMF, this will result in an absorption rate of about 1.2 g of DMF per hour.

An initial dose of about 15 mg/kg of DMT is especially preferred.

It is anticipated that long term treatment of about 2 years with DMF would be required, before the production of the Ksp37 protein is sufficiently genetically modified to the therapeutic range required for at least 20 years.

The invention therefore identifies the optimum concentration range within which Ksp37, having a molecular weight in the range ranging from 24 kDa to 45 kDa, enhances the immune response of a subject against viral infection and/or diseases associated with immune system disorders and/or viral cancers, and provides for the preparation of medicines and medicaments for the treatment of viral infection and/or diseases associated with immune system disorders and/or viral cancers and a method of treatment and/or protection against viral infection and/or diseases associated with immune system disorders and/or viral cancers.

All publications cited herein are incorporated by reference in their entirety. Citation or discussion of a reference herein shall not be construed as an admission that such is prior art to the present invention.

1. A pharmaceutical substance comprising a therapeutically effective amount of one or more of a clinically modified or genetically engineered Ksp37 protein and/or a vector encoded with a KSP37 gene which will translate to a Ksp37 protein and/or a polar compound selected from the group including N,N-dimethylformamide (DMF), for use in enhancing the immune response of a subject against viral infection and/or diseases associated with immune system disorders and/or viral cancers, wherein the substance is formulated to raise the Ksp37 blood plasma concentration level of the subject to between 400 ng/mL and 700 ng/mL when administered to the subject one or more times over a suitable period.

2. (canceled)

3. The pharmaceutical substance as claimed in claim 1, wherein the substance includes a pharmaceutically accepted excipient.

4. The pharmaceutical substance as claimed in claim 1, wherein the vector encoded with a KSP37 gene contains a nucleic acid sequence that translates to a protein identical to the naturally occurring Ksp37 protein.

5. The pharmaceutical substance as claimed in claim 1, wherein the vector encoded with the KSP37 gene which will translate to a Ksp37 protein includes a pGEM-T vector or a pCMV3-C-GFPSpark.

6. The pharmaceutical substance as claimed in claim 5, wherein the vector is pGEM-T vector, KSP37/FGFBP2 cDNA ORF CLONE (SinoBiological).

7. (canceled)

8. The pharmaceutical substance as claimed in claim 1, wherein the DMF activates the Ksp37 production and wherein the amount of DMF for activating Ksp37 production is a dose that results in a peak plasma level of 2 mg/l-200 mg/l, more preferably 100 mg/l-200 mg/l, and most preferably 100 mg/l-150 mg/l or 150 mg/l-200 mg/l of DMF.

9-18. (canceled)

19. A method of protecting a subject from disease characterised by viral infection and/or diseases associated with immune system disorder and viral cancers, and/or a method of treating a subject suffering from disease characterised by viral infection and/or diseases associated with immune system disorder and/or viral cancers, the method comprising administering to the subject a therapeutically effective amount of the pharmaceutical substance of claim 1 to increase the blood plasma levels of Ksp37 protein in the subject to between 400 ng/mL and 700 ng/mL.

20. The method as claimed in claim 19, wherein the therapeutically effective amount of clinically modified or genetically engineered Ksp37 protein and/or proteins is between 0.001 μg/kg body weight of the subject microgram and 20 μg/kg body weight of the subject.

21. The method as claimed in claim 19, wherein the pharmaceutical substance is administered to the subject via an acceptable administration route, including nasal, oral, topical, inhalation, transdermal, rectal or parenteral administration.

22. The method as claimed in claim 19, wherein the Ksp37 protein is extracted from blood components and/or tissue, then purified, acetylised, genetically engineered, cloned and transferred back to a mammalian host as a therapeutic and/or preventative vaccine against viral infection and/or diseases associated with immune system disorders and/or viral cancers.

23. The method as claimed in claim 19, wherein the therapeutically effective amount of DMF for activating Ksp37 production is a dose that results in a peak plasma level of 2 mg/l-200 mg/l, preferably 100 mg/l-200 mg/l, and most preferably 100 mg/l-150 mg/l or 150 mg/l-200 mg/l of DMF.

24. The method as claimed in claim 23, wherein the DMF is administered transdermal.

25. The method as claimed in claim 19, wherein the virus is a retrovirus, specifically HIV.

26. The method as claimed in claim 19, wherein the viral cancer is ovarian cancer or leukaemia.

27. The method as claimed in claim 19, wherein the subject is a mammal.

28. A therapeutic and/or preventative vaccine against viral infection and/or diseases associated with immune system disorders and/or viral cancers, the vaccine comprising a clinically modified or genetically engineered Ksp37 protein and/or proteins and/or a vector encoded with a KSP37 gene which will translate to a Ksp37 protein and/or proteins.
